(19) United States
(12) Patent Application Publication
(10) Pub. No.: US 2023/0192638 A1
(43) Pub. Date: Jun. 22, 2023

(54) POLYPHENOLS DERIVATIVES

(71) Applicant: Epirium Bio Inc., San Diego, CA (US)

(72) Inventors: Sohail AHMAD, Uttar Pradesh (IN); Ajay Ramchandra TILEKAR, Maharashtra (IN); Bhavesh DAVE, Maharashtra (IN)

(21) Appl. No.: 18/082,401

(22) Filed: Dec. 15, 2022

Related U.S. Application Data
(60) Provisional application No. 63/265,681, filed on Dec. 17, 2021.

(51) Int. Cl. C07D 311/28

(52) U.S. Cl. CPC C07D 311/28

(57) ABSTRACT

The present application discloses derivatives of polyphenols, pharmaceutically acceptable salts, stereoisomers or tautomers thereof. The compounds and compositions described herein can be used in therapy.

### CROSS-REFERENCE TO RELATED APPLICATIONS

This application claims the benefit of provisional U.S. Appl. Ser. No. 63/265,681, filed Dec. 17, 2021, which is incorporated herein in its entirety for all purposes.

### FIELD OF THE INVENTION

The present disclosure relates generally to derivatives of polyphenols. Process for preparation of the compounds, compositions comprising the compounds and methods of use are also provided.

### BACKGROUND OF THE INVENTION

Polyphenolic natural products are of current interest because of their various biological activities, their occurrence in foodstuffs, and hence their relevance for human health. Polyphenolic natural products have two or more hydroxyl groups on their aromatic rings. Though such polyphenols including catechins and epicatechin are used widely, they have certain drawbacks such as low potency, undesirable pharmacodymanics and pharmacokinetic profile. Hence there is a need to develop new derivatives of polyphenols for improving the physicochemical properties of pharmaceutical and nutraceutical ingredients.

### SUMMARY OF THE INVENTION

Provided herein are compounds, salts thereof, pharmaceutical compositions of the foregoing and methods of making and using the same.

In one aspect, provided is a compound of formula (I):

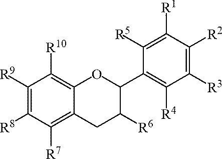

or a pharmaceutically acceptable salt thereof, wherein R¹, R², R³, R⁴, R⁵, R⁶, R⁷, R⁸, R⁹ and R¹⁰ are as detailed herein.

Also provided herein is a pharmaceutical composition comprising a compound of any formula herein, including formula (I), a pharmaceutically acceptable salt thereof, and a pharmaceutically acceptable carrier.

In yet another aspect, the compounds described herein have 5′ AMP-activated protein kinase (AMPK) activities and can be useful in AMPK activation.

### DETAILED DESCRIPTION

The present application discloses derivatives of polyphenols, pharmaceutically acceptable salts, stereoisomers or tautomers thereof, and processes for preparation thereof. The compounds and compositions described herein can be used in therapy.

### Definitions

As used herein and in the appended claims, the singular forms “a”, “an” and “the” include plural forms, unless the context clearly dictates otherwise.

“Alkyl” as used herein refers to and includes, unless otherwise stated, a saturated linear (i.e., unbranched) or branched univalent hydrocarbon chain or combination thereof, having the number of carbon atoms designated (i.e., C₁-C₁₀ means one to ten carbon atoms). Particular alkyl groups are those having 1 to 20 carbon atoms (a “C₁-C₂₀ alkyl”), having 1 to 10 carbon atoms (a “C₁-C₁₀ alkyl”), having 6 to 10 carbon atoms (a “C₆-C₁₀ alkyl”), having 1 to 6 carbon atoms (a “C₁-C₆ alkyl”), having 2 to 6 carbon atoms (a “C₂-C₆ alkyl”), or having 1 to 4 carbon atoms (a “C₁-C₄ alkyl”). Examples of alkyl groups include, but are not limited to, groups such as methyl, ethyl, n-propyl, isopropyl, n-butyl, t-butyl, isobutyl, sec-butyl, n-pentyl, n-hexyl, n-heptyl, n-octyl, n-nonyl, n-decyl, and the like.

As used herein, “therapeutically effective amount” indicates an amount that results in a desired pharmacological and/or physiological effect for the condition. The effect may be prophylactic in terms of completely or partially preventing a condition or symptom thereof and/or may be therapeutic in terms of a partial or complete cure for the condition and/or adverse effect attributable to the condition.

As used herein, the term “pharmaceutically acceptable excipient,” and cognates thereof, refers to adjuvants, binders, diluents, etc. known to the skilled artisan that are suitable for administration to a subject (e.g., a mammal or non-mammal). Combinations of two or more excipients are also contemplated. The pharmaceutically acceptable excipient(s) and any additional components, as described herein, should be compatible for use in the intended route of administration (e.g., oral, parenteral) for a particular dosage form, as would be recognized by the skilled artisan.

“Pharmaceutically acceptable salts” are those salts which retain at least some of the biological activity of the free (non-salt) compound and which can be administered as drugs or pharmaceuticals to a subject. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound of the invention in its free acid or base form with a suitable organic or inorganic base or acid, respectively, and isolating the salt thus formed during subsequent purification.

As used herein, and unless otherwise specified, the terms “about” and “approximately,” when used in connection with doses, amounts, molar percent, or weight percent of ingredients of a composition or a dosage form, mean a dose, amount, molar percent, or weight percent that is recognized by those of ordinary skill in the art to provide a pharmacological effect equivalent to that obtained from the specified dose, amount, molar percent, or weight percent. Specifically, the terms “about” and “approximately,” when used in this context, contemplate a dose, amount, molar percent, or weight percent within 15%, within 10%, within 5%, within 4%, within 3%, within 2%, within 1%, or within 0.5% of the specified dose, amount, molar percent, or weight percent.

Asymmetric centers exist in the compounds disclosed herein. These centers are designated by the symbols “R” or “S,” depending on the configuration of substituents around the chiral carbon atom. It should be understood that stereochemical isomeric forms, including diastereomeric, enantiomeric, and epimeric forms as well as d-isomers and 1-isomers, and mixtures thereof are encompassed. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. All cis, trans, syn, anti, entgegen (E), and zusammen (Z) isomers as well as the appropriate mixtures thereof are included. Additionally, compounds may exist as tautomers; all tautomeric isomers are provided. Additionally, the compounds disclosed herein can exist in unsolvated as well as solvated forms with pharmaceutically acceptable solvents such as water, ethanol, and the like. In general, the solvated forms are considered equivalent to the unsolvated forms.

The terms “treat,” “treating,” and “treatment” are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. Often, the beneficial effects that a subject derives from a therapeutic agent do not result in a complete cure of the disease, disorder or condition.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

### Compounds

In one aspect, provided is a compound of formula (I):

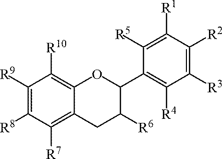

or a pharmaceutically acceptable salt, stereoisomer or tautomer thereof, wherein:
R¹, R², R³, R⁴ and R⁵ are independently H, OH, halo, C₁-C₆ alkyl, or C₁-C₆ alkoxy;
R⁶ is OH or H; and
R⁷, R⁸, R⁹ and R¹⁰ are independently H, OH, C₁-C₆ alkyl, or C₁-C₆ alkoxy,
provided that the compound is not selected from the group consisting of:
2-(3,4-dihydroxyphenyl)chroman-3,7-diol,
2-(3,4-dihydroxyphenyl)chroman-3,5-diol,
2-(3-hydroxyphenyl)chroman-3,5,7-triol,
2-(3,4-dihydroxy-2-methylphenyl)chroman-3,5,7-triol,
2-(2-fluoro-3,4-dihydroxyphenyl)chroman-3,5,7-triol,
2-(2-fluoro-4,5-dihydroxyphenyl)chromane-3,5,7-triol,
2-(3-fluoro-4-hydroxyphenyl)chromane-3,5,7-triol,
2-(3,4-dihydroxy-5-methylphenyl)chromane-3,5,7-triol,
2-(3,4-dihydroxyphenyl)chromane-3,5,7-triol,
4-(3-hydroxychroman-2-yl)benzene-1,2-diol,
2-(3-hydroxyphenyl)chromane-3,5-diol,
2-(3-hydroxyphenyl)chromane-3,7-diol,
2-(3-hydroxyphenyl)chroman-3-ol,
2-(3-methoxyphenyl)chromane-3,7-diol,
2-(3-hydroxyphenyl)-7-methoxychroman-3-ol,
Cis (±) 7-methoxy-2-(3-methoxyphenyl)chroman-3-ol,
Cis (±) 7-methoxy-2-(4-methoxyphenyl)chroman-3-ol,
2-(3-methoxy-4-methylphenyl)chromane-3,7-diol,
2-(3-hydroxy-4-methylphenyl)chromane-3,7-diol,
2-(4-fluoro-3-methoxyphenyl)chromane-3,7-diol,
2-(4-fluoro-3-hydroxyphenyl)chromane-3,7-diol,
2-(3,4-dihydroxyphenyl)chroman-3,7-diol,
2-(3,4-dihydroxyphenyl)chroman-3,5-diol,
2-(3-ethoxy-4-hydroxyphenyl)chromane-3,5,7-triol,
4-(3-hydroxy-5,7-dimethoxychroman-2-yl)benzene-1,2-diol,
2-(4-hydroxy-3-propoxyphenyl)chromane-3,5,7-triol,
2-(4-ethoxy-3-hydroxyphenyl)chromane-3,5,7-triol,
2-(3-hydroxy-4-propoxyphenyl)chromane-3,5,7-triol, and
2-(4-hydroxy-3-methoxyphenyl)chromane-3,5,7-triol,
or a pharmaceutically acceptable salt, stereoisomer or tautomer thereof.

In some embodiments, the substitution at C2 and C3 of the pyran ring is cis(+) or cis(-) or a mixture thereof.

In some embodiments, a compound of formula (I) is a compound of formula (I-a),

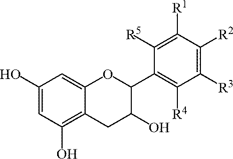

or a pharmaceutically acceptable salt, stereoisomer or tautomer thereof, wherein the substitution at C2 and C3 of pyran ring is cis(+) or cis(-) or a mixture thereof.

In some embodiments, R¹, R², R³, R⁴ and R⁵ are independently H, OH, F, Cl, CH₃, CF₃, or OCH₃. In some embodiments, R¹ is H. In some embodiments, R¹ is OH. In some embodiments, R¹ is C₁-C₆ alkoxy. In some embodiments, R¹ is methoxy. In some embodiments, R¹ is C₁-C₆ alkyl. In some embodiments, R¹ is methyl. In some embodiments, R¹ is halo. In some embodiments, R¹ is fluoro. In some embodiments, at least one of R¹, R⁴ and R⁵ is OH, halo, C₁-C₆ alkyl, or C₁-C₆ alkoxy.

In some embodiments, R² is H. In some embodiments, R² is halo. In some embodiments, R² is fluoro. In some embodiments, R³ is H. In some embodiments, R³ is OH. In some embodiments, R³ is C₁-C₆ alkoxy. In some embodiments, R³ is methoxy. In some embodiments, R³ is C₁-C₆ alkyl. In some embodiments, R³ is methyl. In some embodiments, R³ is halo. In some embodiments, R³ is fluoro. In some embodiments, R⁴ is H. In some embodiments, R⁴ is halo. In some embodiments, R⁴ is fluoro. In some embodiments, R⁴ is chloro. In some embodiments, R⁵ is H. In some embodiments, R⁵ is halo. In some embodiments, R⁵ is fluoro. In some embodiments, R⁵ is chloro.

In some embodiments, R³ is OH, and R⁵ is halo. In some embodiments, R³ is OH, and R⁵ is fluoro. In some embodiments, R¹ is OH, and R² is halo. In some embodiments, R¹ is OH, and R² is fluoro. In some embodiments, R¹ is OH, and R³ is halo. In some embodiments, R¹ is OH, and R³ is fluoro. In some embodiments, R¹ is OH, and R⁵ is halo. In some embodiments, R¹ is OH, and R⁵ is chloro. In some embodiments, R¹ is OH, and R³ is C₁-C₆ alkyl. In some embodiments, R¹ is OH, and R³ is methyl. In some embodiments, R¹ is C₁-C₆ alkoxy, and R² is OH. In some embodiments, R¹ is methoxy, and R² is OH. In some embodiments, R¹ and R² are each OH.

In some embodiments, R⁶ is OH. In some embodiments, R⁷ and R⁹ are independently OH or OCH₃, and R⁸ and R¹⁰ are H. In some embodiments, R⁷ is OH. In some embodiments, R⁷ is H. In some embodiments, R⁸ is H. In some embodiments, R⁹ is OH. In some embodiments, R¹⁰ is H. In some embodiments, R¹⁰ is C₁-C₆ alkyl. In some embodiments, R¹⁰ is methyl. In some embodiments, R⁷ and R⁹ are each OH. In some embodiments, R⁸ and R¹⁰ are each H. In some embodiments, R⁷ and R⁹ are each OH, and R⁸ and R¹⁰ are each H. In some embodiments, R⁷ is H, and R⁹ is OH. In some embodiments, R⁸ is H, and R¹⁰ is C₁-C₆ alkyl. In some embodiments, R⁸ is H, and R¹⁰ is methyl. In some embodiments, R⁷ and R⁸ are each H, R⁹ is OH, and R¹⁰ is C₁-C₆ alkyl. In some embodiments, R⁷ and R⁸ are each H, R⁹ is OH, and R¹⁰ is methyl.

In some embodiments, a compound of formula (I) is selected from the group consisting of:

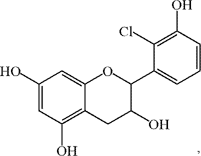

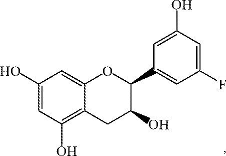

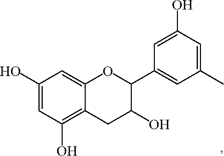

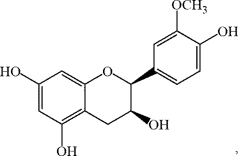

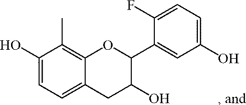

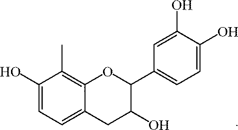

In some embodiments, a compound of formula (I) is not selected from the group consisting of 2-(3,4-dihydroxyphenyl)chroman-3,7-diol, 2-(3,4-dihydroxyphenyl)chroman-3,5-diol, 2-(3-hydroxyphenyl)chroman-3,5,7-triol, 2-(3,4-dihydroxy-2-methylphenyl)chroman-3,5,7-triol, 2-(2-fluoro-3,4-dihydroxyphenyl)chroman-3,5,7-triol, 2-(2-fluoro-4,5-dihydroxyphenyl)chromane-3,5,7-triol, 2-(3-fluoro-4-hydroxyphenyl)chromane-3,5,7-triol, 2-(3,4-dihydroxy-5-methylphenyl)chromane-3,5,7-triol, 2-(3,4-dihydroxyphenyl)chromane-3,5,7-triol, 4-(3-hydroxychroman-2-yl)benzene-1,2-diol, 2-(3-hydroxyphenyl)chromane-3,5-diol, 2-(3-hydroxyphenyl)chromane-3,7-diol, 2-(3-hydroxyphenyl)chroman-3-ol, 2-(3-methoxyphenyl)chromane-3,7-diol, 2-(3-hydroxyphenyl)-7-methoxychroman-3-ol, Cis (±) 7-methoxy-2-(3-methoxyphenyl)chroman-3-ol, Cis (±) 7-methoxy-2-(4-methoxyphenyl)chroman-3-ol, 2-(3-methoxy-4-methylphenyl)chromane-3,7-diol, 2-(3-hydroxy-4-methylphenyl)chromane-3,7-diol, 2-(4-fluoro-3-methoxyphenyl)chromane-3,7-diol, 2-(4-fluoro-3-hydroxyphenyl)chromane-3,7-diol, 2-(3,4-dihydroxyphenyl)chroman-3,7-diol, 2-(3,4-dihydroxyphenyl)chroman-3,5-diol, 2-(3-ethoxy-4-hydroxyphenyl)chromane-3,5,7-triol, 4-(3-hydroxy-5,7-dimethoxychroman-2-yl)benzene-1,2-diol, 2-(4-hydroxy-3-propoxyphenyl)chromane-3,5,7-triol, 2-(4-ethoxy-3-hydroxyphenyl)chromane-3,5,7-triol, 2-(3-hydroxy-4-propoxyphenyl)chromane-3,5,7-triol, and 2-(4-hydroxy-3-methoxyphenyl)chromane-3,5,7-triol, or a pharmaceutically acceptable salt, stereoisomer or tautomer thereof.

Representative compounds are listed in Table 1.

| Compound | Name |
|---|---|
| 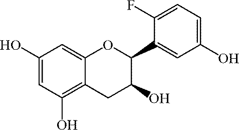 [SMILES: Oc1cc(O)c2c(c1)O[C@@H](c1cc(O)ccc1F)[C@@H](O)C2] | (2S,3S)-2-(2-fluoro-5-hydroxyphenyl)chromane-3,5,7-triol |
| 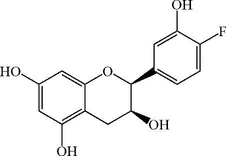 [SMILES: Oc1cc(O)c2c(c1)O[C@@H](c1ccc(F)c(O)c1)[C@@H](O)C2] | (2S,3S)-2-(4-fluoro-3-hydroxyphenyl)chromane-3,5,7-triol |
| 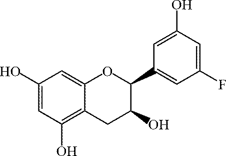 [SMILES: Oc1cc(F)cc([C@@H]2Oc3cc(O)cc(O)c3C[C@@H]2O)c1] | 2-(3-fluoro-5-hydroxyphenyl)chromane-3,5,7-triol |
| 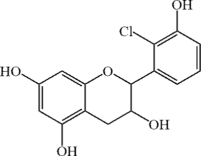 [SMILES: Oc1cc(O)c2c(c1)OC(c1cccc(O)c1Cl)C(O)C2] | 2-(2-chloro-3-hydroxyphenyl)chromane-3,5,7-triol |
| 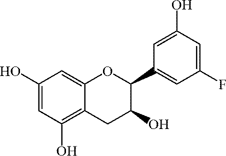 [SMILES: Oc1cc(F)cc([C@@H]2Oc3cc(O)cc(O)c3C[C@@H]2O)c1] | 2-(3-fluoro-5-hydroxyphenyl)chromane-3,5,7-triol |
| 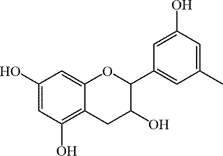 [SMILES: Cc1cc(O)cc(C2Oc3cc(O)cc(O)c3CC2O)c1] | 2-(3-hydroxy-5-methylphenyl)chromane-3,5,7-triol |
| 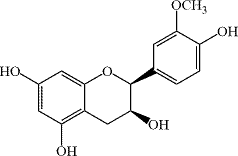 [SMILES: COc1cc([C@@H]2Oc3cc(O)cc(O)c3C[C@@H]2O)ccc1O] | (2S,3S)-2-(4-hydroxy-3-methoxyphenyl)chromane-3,5,7-triol |
| 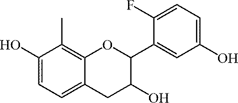 [SMILES: Cc1c(O)ccc2c1OC(c1cc(O)ccc1F)C(O)C2] | 2-(2-fluoro-5-hydroxyphenyl)-8-methylchromane-3,7-diol |
| 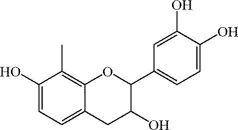 [SMILES: Cc1c(O)ccc2c1OC(c1ccc(O)c(O)c1)C(O)C2] | 2-(3,4-dihydroxyphenyl)-8-methylchromane-3,7-diol |

Provided herein is a compound selected from the group consisting of the compounds described in Table 1.

Also provided herein are, where applicable, any and all stereoisomers of the compounds depicted herein, including geometric isomers (e.g., cis/trans isomers or E/Z isomers), enantiomers, diastereomers, or mixtures thereof in any ratio, including racemic mixtures.

Compositions of any of the compounds detailed herein are embraced by this disclosure. In some embodiments, the present invention includes pharmaceutical compositions comprising a compound as detailed herein or a salt thereof and a pharmaceutically acceptable carrier or excipient.

Compounds described herein have 5′ AMP-activated protein kinase (AMPK) activities and can be useful in AMPK activation. In some embodiments, the present invention includes a method of preventing and/or treating such a disease or disorder in a subject in need thereof comprising administering to the subject a therapeutically effective amount of a compound as detailed herein, including but not limited to a compound of formula (I), a pharmaceutically acceptable salt thereof, or a pharmaceutical composition comprising such compound or salt.

In the descriptions herein, it is understood that every description, variation, embodiment or aspect of a moiety may be combined with every description, variation, embodiment or aspect of other moieties the same as if each and every combination of descriptions is specifically and individually listed. For example, every description, variation, embodiment or aspect provided herein with respect to X¹ of formula (I) may be combined with every description, variation, embodiment or aspect of X², X³, X⁴, R⁴, X⁵, and/or R⁶ the same as if each and every combination were specifically and individually listed. Every description, variation, embodiment or aspect provided herein with respect to X of formula (II) may be combined with every description, variation, embodiment, or aspect of R⁷, R⁸, and/or R⁹ the same as if each and every combination were specifically and individually listed. Every description, variation, embodiment or aspect provided herein with respect to R¹⁵ of formula (III) may be combined with every description, variation, embodiment, or aspect of R¹⁶, R¹⁷, and/or R¹⁸ the same as if each and every combination were specifically and individually listed.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. Unless otherwise stated, “substantially pure” intends a composition that contains no more than 35% impurity, wherein the impurity denotes a compound other than the compound comprising the majority of the composition or a salt thereof. In some embodiments, a composition of substantially pure compound or a salt thereof is provided wherein the composition contains no more than 25%, 20%, 15%, 10%, or 5% impurity. In some embodiments, a composition of substantially pure compound or a salt thereof is provided wherein the composition contains or no more than 3%, 2%, 1% or 0.5% impurity. In some embodiments, the provided compounds are sterilized.

Also provided herein are, where applicable, are any and all stereoisomers of the compounds depicted herein, including compounds of formulae (I), (II), and (III), including geometric isomers (e.g., cis/trans isomers or E/Z isomers), enantiomers, diastereomers, or mixtures thereof in any ratio, including racemic mixtures. In some embodiments, a provided compound has two stereocenters which are in the cis configuration. In some embodiments, a provided compound has two stereocenters which are in the trans configuration. In some embodiments, a provided compound has two stereocenters in the (S, S) configuration. In some embodiments, a provided compound has two stereocenters in the (R, R) configuration. In some embodiments, a provided compound has two stereocenters in the (S, R) configuration. In some embodiments, a provided compound has two stereocenters in the (R, S) configuration. In some embodiments, a provided compound is present at 75% stereoisomeric purity in a mixture of enantiomers and/or diastereomers. In some embodiments, a provided compound is present at 80% stereoisomeric purity in a mixture of enantiomers and/or diastereomers. In some embodiments, a provided compound is present at 90% stereoisomeric purity in a mixture of enantiomers and/or diastereomers. In some embodiments, a provided compound is present at 95% stereoisomeric purity in a mixture of enantiomers and/or diastereomers. In some embodiments, a provided compound is present at 99% stereoisomeric purity in a mixture of enantiomers and/or diastereomers. In some embodiments, a provided compound is present at 99.5% stereoisomeric purity in a mixture of enantiomers and/or diastereomers. In some embodiments, a provided compound is present at 99.9% stereoisomeric purity in a mixture of enantiomers and/or diastereomers. In some embodiments, a provided compound is a single stereoisomer that is substantially free of other enantiomers and/or diastereomers.

It is understood that compounds with tautomeric forms are described and embraced herein. Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein.

The compounds described here also intend isotopically-labeled and/or isotopically-enriched forms. The compounds herein may contain unnatural proportions of atomic isotopes at one or more of the atoms that constitute such compounds. In some embodiments, the compound is isotopically-labeled, such as an isotopically-labeled compound of the formula (I) or variations thereof described herein, where a fraction of one or more atoms are replaced by an isotope of the same element. Exemplary isotopes that can be incorporated into the provided compounds include isotopes of hydrogen, carbon, nitrogen, oxygen, phosphorus, sulfur, chlorine, such as ²H, ³H, ¹¹C, ¹³C, ¹⁴C ¹³N, ¹⁵O, ¹⁷O, ³²P, ³⁵S, ¹⁸F, ³⁶Cl. Certain isotope labeled compounds (e.g. ³H and ¹⁴C) is useful in compound or substrate tissue distribution studies. Incorporation of heavier isotopes such as deuterium (²H) can afford certain therapeutic advantages resulting from greater metabolic stability, for example, increased in vivo half-life, or reduced dosage requirements and, hence may be preferred in some instances.

Isotopically-labeled compounds can generally be prepared by standard methods and techniques known to those skilled in the art or by procedures similar to those described in the accompanying Examples substituting appropriate isotopically-labeled reagents in place of the corresponding non-labeled reagent.

The provided compounds may be prepared by a number of processes, including but not limited to the processes generally described below. In the following process descriptions, the symbols when used in the formulae depicted are to be understood to represent those groups described above in relation to the formulae herein.

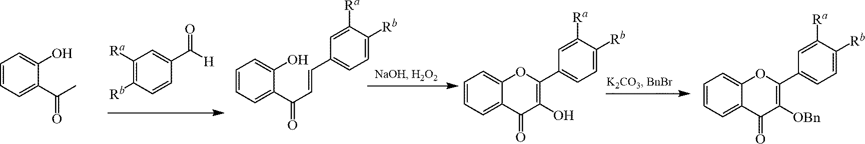

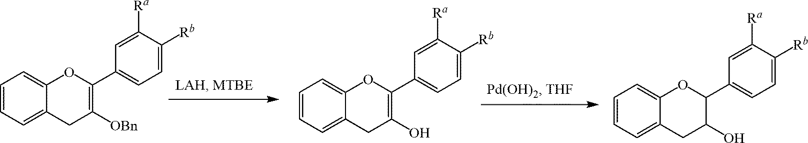

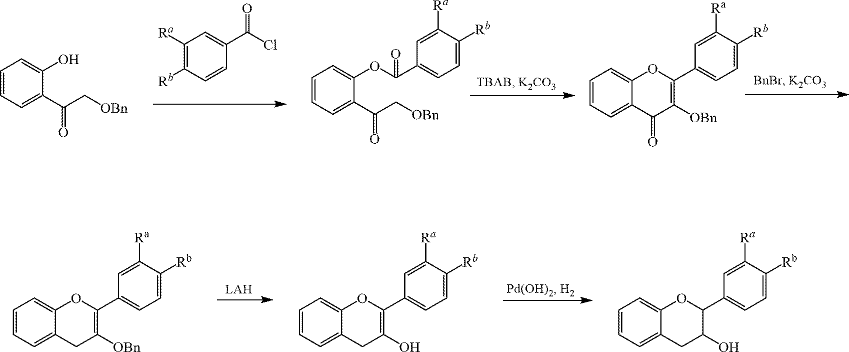

### Methods of Use

The provided compounds and compositions can be used to improve the physicochemical properties of pharmaceutical and nutraceutical ingredients.

The compounds and compositions described herein may be used for all indications in which epicatechin is indicated, including, without limitation, any of the diseases or conditions described in WO2012/170430, WO2013/022846, WO2013/142816, US2018/0193306, WO2014/162320, WO2017/221269, and WO2018/083713, each of which is hereby incorporated by reference in its entirety.

In yet another aspect, provided is methods for treating diseases or disorders that would benefit from increased expression of Electron transfer Chain (ETC), particularly ETC IV. The methods involve administering to a subject in need thereof a therapeutically effective amount of the compounds and compositions described herein.

The vast majority of the body’s need for ATP is supplied through the process of oxidative phosphorylation, carried out in the mitochondria in all tissues. There are 5 protein complexes, known as the Electron Transport Complexes that effect ATP synthesis. ETC I, II, III, and IV mediate electron transport. ETC I, III, and IV also function as proton pumps that maintain an electrochemical gradient necessary for activity of ETC V, the ATP synthase enzyme that makes ATP from ADP. Complex Γ, also known as cytochrome c oxidase, (COX), consists of 14 subunits whose assembly into a functional complex requires an additional 30 protein factors. ETC IV is particularly important to oxidative phosphorylation. It is the only one of the ETC complexes to manifest tissue -specific and developmentally regulated isoforms, allowing precise regulation of oxidative phosphorylation under a variety of metabolic demands. Thus the ETC IV (COX) protein complex is considered to be the rate-limiting step in oxidative phosphorylation. Small positive or negative changes in ETC IV can exert a significant impact on health. Selective activation of COX activity has been associated with improved cognition, improved neuronal cell survival under stress, and improved wound healing. Mutations in the numerous proteins that comprise or regulate the activity of ETC IV reveal the pathological consequences of even modest decreases in ETC IV activity. As little as a 30% reduction in COX activity has been shown to induce cardiomyopathy or be associated with the development of neurodegenerative diseases such as Alzheimer’s. Decreases in COX (ETCIV) expression due to mutations or molecular manipulation have been associated with loss of muscle endurance and speed, muscle dystonia, immunodeficiency states due to impaired T cell maturation, cardiomyopathy, particularly of the aging phenotype, ataxia, neurodegeneration, increased toxicity in the setting of ischemia, pulmonary inflammation and fibrosis, encephalopathy, vascular insufficiency, and stimulation of cancer cell proliferation. Additional specific diseases associated with COX subunit isoform mutations causing loss of function include exocrine pancreatic insufficiency, inflammatory lung disease, Charcot-Marie-Tooth disease, infantile encephalomyopathy, and Leigh syndrome neurodegeneration with epilepsy.

The following conditions associated with loss of COX expression or function would be expected to be therapeutically responsive to a potent, preferential inducer of COX (ETC IV) expression: impaired cognition, neurodegenerative diseases such as Alzheimer’s or Leigh syndrome, dystonia, sarcopenia, cardiomyopathy of aging or other diseases associated with mitochondrial dysfunction, ischemic vascular disease, immunodeficiency states, ataxia, pulmonary inflammation and fibrosis, infantile encephalomyopathy, epilepsy, Charcot-Marie-Tooth disease, exocrine pancreatic insufficiency, impaired wound healing, growth of cancer cells.

In some embodiments, the compounds and compositions described herein may be used for inducing mitochondrial biogenesis, including biogenesis of any one or more of ETC I, II, III, IV, and V.

In addition, epicatechin can be used in lowering the elevated triglycerides. In some embodiments, the compounds and compositions described herein may be use in medicament for conditions associated with elevated triglycerides, such as metabolic syndrome, Type II diabetes, congenital hyperlipidemias, and drug-induced hyperlipidemia, as is observed with corticosteroid treatments.

In another embodiment, provided are methods for prophylactic and/or therapeutic treatment of conditions related to mitochondrial dysfunction resulting from administration of one or more chemical compositions that exhibit mitochondrial toxicity. In some embodiments, the mitochondrial toxicity is identified based on or associated with one or more biological effects, which include, but are not limited to, abnormal mitochondrial respiration, abnormal oxygen consumption, abnormal extracellular acidification rate, abnormal mitochondrial number, abnormal lactate accumulation, and abnormal ATP levels. In some embodiments, the mitochondrial toxicity is identified based on or associated with one or more physiological manifestations, which include, but are not limited to, elevations in markers known to relate to injury to the heart, liver, and/or kidney, elevated serum liver enzymes, elevated cardiac enzymes, lactic acidosis, elevated blood glucose, and elevated serum creatinine. In another embodiment, provided are methods for treating chronic mitochondrial depletion and the symptoms arising as a result of drug-associated toxicity or as a combination of drug associated toxicity occurring within a background of biological depletion of mitochondrial number, as occurs in diabetes, obesity, and during the course of aging. In another embodiment, provided are methods for treating chronic perturbation of mitochondria function or structure, including chronic myopathy, sarcopenia, persistent diabetes, chronic fatigue syndromes, gastrointestinal symptoms, liver, and cardiovascular dysfunction and failure, neurological symptoms, impaired sleep, and persistent alteration in cognitive acuity or function, such as memory.

In another embodiment, provided are methods for treating, preventing, or reversing injury to skeletal or cardiac muscles, for treating or preventing diseases relating to the structure and function of skeletal or cardiac muscles, and for inducing regeneration or restructuring of skeletal or cardiac muscle as a means for treating disease relating to abnormalities in the skeletal or cardiac muscle structure and function in a subject.

In some embodiments, provided are methods for treatment of impaired skeletal or cardiac muscle function due to aging, obesity, disuse or inactivity, exposure to potentially toxic nutritional agents such as fructose, or exposure to inadequate nutrition such as starvation or malnutrition.

In some embodiments, provided are methods for the treatment of muscle-related side effects of athletic training or competition including soreness, cramping, weakness, pain, or injury.

In some embodiments, provided are methods for the treatment of skeletal or cardiac muscle diseases associated with ischemia, or impaired or inadequate blood flow. In some embodiments, the diseases are selected from the group consisting of atherosclerosis, trauma, diabetes, vascular stenosis, peripheral arterial disease, vasculopathy, and vasculitis.

In some embodiments, provided are methods for the treatment of diseases associated with genetic disorders that directly or indirectly affect the number, structure, or function of cardiac muscle cells or skeletal muscle cells. In some embodiments, the disease is selected from the group consisting of muscular dystrophies and Friedreich’s ataxia.

In some embodiments, provided are methods for the treatment of diseases associated with impaired neurological control of muscular activity resulting in consequent abnormalities in structure and function of skeletal muscles due to inactivity, aberrant contractility, or contracted states. In some embodiments, the disease is selected from the group consisting of peripheral denervation syndromes, trauma, amyotrophic lateral sclerosis, meningitis, and structural abnormalities of the spine, whether congenital or acquired.

In some embodiments, provided are methods for the treatment of diseases associated with loss of number, loss of function, or loss of correct, optimally efficient internal organization of skeletal muscle cells or cardiac muscle cells. In some embodiments, the disease is muscle wasting. In some embodiments, the disease is sarcopenia. In some embodiments, sarcopenia is associated with a variety of disorders, including aging, diabetes, abnormal metabolic conditions, infection, inflammation, autoimmune disease, cardiac dysfunction, arthritis congestive heart failure, aging, myocarditis, myositis, polymyalgia rheumatica, polymyositis, HIV, cancer, side effects of chemotherapy, malnutrition, aging, inborn errors of metabolism, trauma, stroke, and neurological impairment.

In some embodiments, the method of treating diseases associated with loss of number, loss of function, or loss of correct, optimally efficient internal organization of skeletal muscle cells or cardiac muscle cells further comprises exercise or programmatic sequences or intensities of exercise.

In some embodiments, provided are methods for enhancing sports performance, endurance, building muscle shape or strength, or facilitating recovery from the effects of training or competition.

In some embodiments, provided are methods for treating muscle injury, weakness, or pain associated with the administration of medicines. In some embodiments, provided are methods for use to prevent, ameliorate, or reverse muscle injury associated with medicines that damage mitochondria and/or cause myopathy as a secondary consequence.

In some embodiments of any one of the embodiments disclosed above, the skeletal or cardiac muscle injury of dysfunction in the subject is identified based on or associated with one or more physiological manifestations, which include, but are not limited to, elevated plasma levels of cardiac or skeletal muscle enzymes or proteins, such as myoglobin, troponin, or creatine phosphokinase, lactic acidosis, and elevated serum creatinine.

In some embodiments, provided are methods for stimulating the increased number or function of skeletal muscle cells or contractile muscle cells. Such stimulation of muscle cells may comprise stimulation of one or more aspects of muscle cell function, including cell division, muscle cell regeneration, activation of muscle satellite cells and their differentiation into adult muscle cells, recovery from injury, increased number or function of mitochondria or processes serving mitochondrial function, increased expression of proteins contributing to contractility, regulation of biochemical or translational processes, mitoses, or transduction of mechanical energy via dystrophin or other attachment processes. The methods and compositions described herein can assist in prevention of the consequences of muscle injury or dysfunction which have not yet occurred, as well as provide for the active therapy of muscle injury, dysfunction, or diseases which have already occurred.

In some embodiments, provided are methods of using muscle proteins whose expression is stimulated by administration of the compounds and compositions described herein as diagnostic biomarkers by which to determine the time and degree of muscle response to the therapeutic methods and compositions disclosed herein. Such biomarkers may be determined by measuring in tissue, plasma, blood, or urine the proteins themselves or the DNA or RNA nucleotides that encode for the proteins. In one embodiment, a decrease in the body of useful muscle proteins, such as dystrophin, or the presence of inhibitory proteins, such as thromobospondin, may be used to diagnose the severity of the abnormality of cardiac muscle structure or function or the probability of response to the therapeutic methods and compositions described herein. In another embodiment, changes in the levels of such biomarkers may be used to gauge the success or failure of certain therapeutic modalities, including those disclosed herein, in order to optimize the dose and to decide whether to maintain or change therapeutic methods and compositions.

In some embodiments, provided are methods of inducing follistatin production, inhibiting myostatin production, and/or increasing the ratio of follistatin to myostatin. This may be, for example, in associated with treating a muscle or bone considition or disorder.

Additional embodiments disclosed herein relate to a method to induce the increased cellular or muscular or bodily production of follistatin and follistatin-like proteins in order to reverse or ameliorate weakness of bone, thus preventing bone fractures, which may in some instances be caused by administration of compounds known to induce weakness of or damage to bone, impairment of bone generation, or impairment of bone growth, including but not limited to corticosteroids such as prednisone or deflazacort, anticonvulsants such as phenytoin and phenobarbital, chemotherapeutics such as aromatase inhibitors, and progestins. Further methods relate to inducing the increased cellular or muscular or bodily production of follistatin or follistatin-like proteins in order to reverse or ameliorate weakness of bone strength, thus preventing bone fractures, which may in some instances be associated with genetic predisposition, aging, inactive lifestyle, or low estrogen states such as menopause or post oophorectomy; a method to induce the increased cellular or muscular or bodily production of follistatin or follistatin-like proteins in order to reverse or ameliorate weakness of bone caused by medical conditions known to be associated with weakness of, or damage to, bone, impairment of bone generation, or impairment of bone growth, such celiac disease, kidney or liver disease, and immunomodulatory diseases such as systemic lupus erythematosus and rheumatoid arthritis; a method to induce the increased cellular or muscular or bodily production of follistatin or follistatin-like proteins in order to reverse or ameliorate weakness of bone in conjunction with the administration of other agents used to treat osteoporosis including calcium, Vitamin D, and calcitonin, in order to prevent bone fractures; a method to method to induce increased cellular or muscular or bodily production of follistatin or follistatin-like proteins as a therapeutic to accelerate the healing of bone fractures or to increase the degree of recovery from a bone fracture, such as those experienced in accidents. athletics, or combat; and a method to induce increased cellular or muscular or bodily production of follistatin or follistatin-like proteins in order to prevent systemic loss of bone density, and thus prevent subsequent bone fractures, during the recovery period after orthopedic surgery or after the onset of a disease or condition necessitating long periods of bed rest or physical inactivity, which are known to result in decreased bone density and muscle weakness.

In some embodiments, provided are methods for treating or preventing neurodegenerative diseases, such as amyotrophic lateral sclerosis (ALS), Parkinson’s disease, Huntington’s disease, spinal cord injury or abnormality, and peripheral and central neuropathies.

In some embodiments, provided are methods for treating or preventing celiac disease, kidney disease, liver disease, inflammatory diseases such as systemic lupus erythematosus and rheumatoid arthritis, osteoporosis, and bone fracture.

Conditions that may be treated by the compounds, compositions, and methods provided herein include: impaired skeletal and cardiac muscle function, recovery of skeletal or cardiac muscle health or function, functionally significant regeneration of skeletal or cardiac muscle cells or function.

In some embodiments, provided are methods for treating acute coronary syndromes, including but not limited to myocardial infarction and angina; acute ischemic events in other organs and tissues, renal injury, renal ischemia and diseases of the aorta and its branches; injuries arising from medical interventions, including but not limited to coronary artery bypass grafting (CABG) procedures and aneurysm repair; cancer; and metabolic diseases, diabetes mellitus and other such disorders.

In some embodiments, provided are methods for treating or preventing dystrophinopathy, such as Duchenne muscular dystrophy, Becker muscular dystrophy, and DMD-associated cardiomyopathy.

In some embodiments, provided are methods for treating or preventing sarcoglycanopathy, including α-sarcoglycanopathy (LGMD2D), β-sarcoglycanopathy (LGMD2E), γ-sarcoglycanopathy (LGMD2C), 8-sarcoglycanopathy (LGMD2F) and ε-sarcoglycanopathy (myoclonic dystonia). Sarcoglycanopathies include four subtypes of autosomal recessive limb-girdle muscular dystrophy (LGMD2C, LGMD2D, LGMD2E, and LGMD2F) that are caused, respectively, by mutations in the SGCG, SGCA, SGCB, and SGCD genes.

In some embodiments, provided are methods for treating or preventing dysferlinopathy, such as Miyoshi myopathy, scapuloperoneal syndrome, distal myopathy with anterior tibial onset, and elevated level of muscular enzyme CK.

Provided is a method of treating or preventing any of the diseases or conditions described herein in a subject in need thereof, comprising administering to the subject a therapeutically effective amount of the compounds and compositions described herein. Also provided are the compounds and compositions described herein for use the manufacture of a medicament for treating or preventing any of the diseases or conditions described herein in a subject in need thereof. Also provided are the compounds and compositions described herein for use in treating or preventing a disease or condition described herein in a subject in need thereof. Also provided are the compounds and compositions described herein for use in medical therapy. Also provided is use of the compounds and compositions described herein for treating or preventing a disease or condition described herein in a subject in need thereof.

In some aspects, provided herein is a method of treating or preventing a disease or disorder that would benefit from inhibition of ATP hydrolysis in a subject in need thereof, comprising administering to the subject a therapeutically effective amount of a provided compound or a pharmaceutically acceptable salt thereof. In some embodiments, the inhibition of ATP hydrolysis does not block mitochondrial ATP synthesis (e.g., complex V ATP synthesis).

In some embodiments, the disease or disorder causes metabolic dysfunction. In some embodiments, the disease or disorder causes impaired mitochondrial respiration. In some embodiments, the disease or disorder causes mitochondrial toxicity. In some embodiments, the mitochondrial toxicity is identified based on or associated with one or more biological effects, which include, but are not limited to, abnormal mitochondrial respiration, abnormal oxygen consumption, abnormal extracellular acidification rate, abnormal mitochondrial number, abnormal lactate accumulation, and abnormal ATP levels. In some embodiments, the mitochondrial toxicity is identified based on or associated with one or more physiological manifestations, which include, but are not limited to, elevations in markers known to relate to injury to the heart, liver, and/or kidney, elevated serum liver enzymes, elevated cardiac enzymes, lactic acidosis, elevated blood glucose, and elevated serum creatinine. Methods for assessing such biological effects or markers are known in the art and may be used in connection with the embodiments described herein. In some embodiments, the disease or disorder deceases mitochondrial ATP synthesis. In some embodiments, the disease or disorder increases ATP hydrolysis.

In some aspects, provided herein is a method of treating or preventing a condition related to acute or chronic excessive glutamate exposure in a subject in need thereof, comprising administering to the subject a therapeutically effective amount of epicatechin or a pharmaceutically acceptable salt thereof.

As used herein, the phrase “excessive glutamate exposure” refers to an amount of glutamate that induces excessive stimulation of glutamate receptors. In some embodiments, the excessive glutamate exposure results from increased extracellular glutamate concentrations. In some embodiments, the excessive glutamate exposure results from excessive glutamate release from the presynaptic membrane. In some embodiments, the excessive glutamate exposure results from impaired glutamate reuptake function. In some embodiments, the excessive glutamate refers to glutamate levels that are increased by greater than any of about 0.05-fold, 0.1-fold, 0.5-fold, 1-fold, 2-fold, 3-fold, 4-fold, 5-fold, or more, compared to the glutamate levels prior to excitotoxicity. In some embodiments, the excessive glutamate refers to glutamate levels that are increased by less than any of about 5-fold, 4-fold, 3-fold, 2-fold, 1-fold, 0.5-fold, 0.1-fold, 0.05-fold, or less, compared to the glutamate levels prior to excitotoxicity.

In some embodiments, the excessive glutamate exposure causes glutamate excitotoxicity. Glutamate excitotoxicity is the excessive stimulation of glutamate receptors, such as N-methyl-D-aspartate (NMDA) receptors, α-amino-3-hydroxy-5-methyl-4-isoxazolepropionic acid (AMPA) receptors, and/or kainate receptors. In some embodiments, glutamate excitotoxicity may induce neuronal degeneration, such as degeneration of dopaminergic neurons, thereby resulting in motor dysfunction. In some embodiments, the excessive glutamate exposure causes excessive stimulation of glutamate receptors.

In some embodiments, the excessive glutamate exposure increases intracellular sodium (Na²⁺) buildup. In some embodiments, the excessive glutamate exposure induces intracellular calcium (Ca²⁺) buildup. In some embodiments, the intracellular Ca²⁺ and/or Na²⁺ is increased by greater than any of about 0.05-fold, 0.1-fold, 0.5-fold, 1-fold, 2-fold, 3-fold, 4-fold, 5-fold, or more, compared to the intracellular Ca²⁺ and/or Na²⁺ prior to the glutamate exposure. In some embodiments, the intracellular Ca²⁺ and/or Na²⁺ is increased less than about 5-fold, 4-fold, 3-fold, 2-fold, 1-fold, 0.5-fold, 0.1-fold, 0.05-fold, or less, compared to the intracellular Ca²⁺ and/or Na²⁺ prior to the glutamate exposure. In some embodiments, the intracellular Ca²⁺ and/or Na²⁺ buildup may cause neuronal damage. In some embodiments, the intracellular Ca²⁺ and/or Na²⁺ buildup may cause cell death.

In some embodiments, the condition is selected from the group consisting of status epilepticus, neuroinflammatory disorders, pediatric seizure disorders, neuronal exocitoxicity, over activation of the NMDA receptor, post-operative syndromes of cognition loss, and loss of synaptic density.

### Dosages

The compounds and compositions disclosed and/or described herein are administered at a therapeutically effective dosage, e.g., a dosage sufficient to provide treatment for the disease state. While human dosage levels have yet to be optimized for the chemical entities described herein, generally, a daily dose ranges from about 0.01 to 100 mg/kg of body weight; in some embodiments, from about 0.05 to 10.0 mg/kg of body weight, and in some embodiments, from about 0.10 to 1.4 mg/kg of body weight. Thus, for administration to a 70 kg person, in some embodiments, the dosage range would be about from 0.7 to 7000 mg per day; in some embodiments, about from 3.5 to 700.0 mg per day, and in some embodiments, about from 7 to 100.0 mg per day. The amount of the chemical entity administered will be dependent, for example, on the subject and disease state being treated, the severity of the affliction, the manner and schedule of administration and the judgment of the prescribing physician. For example, an exemplary dosage range for oral administration is from about 5 mg to about 500 mg per day, and an exemplary intravenous administration dosage is from about 5 mg to about 500 mg per day, each depending upon the pharmacokinetics.

A daily dose is the total amount administered in a day. A daily dose may be, but is not limited to be, administered each day, every other day, each week, every 2 weeks, every month, or at a varied interval. In some embodiments, the daily dose is administered for a period ranging from a single day to the life of the subject. In some embodiments, the daily dose is administered once a day. In some embodiments, the daily dose is administered in multiple divided doses, such as in 2, 3, or 4 divided doses. In some embodiments, the daily dose is administered in 2 divided doses.

Administration of the compounds and compositions described herein can be via any accepted mode of administration for therapeutic agents including, but not limited to, oral, sublingual, subcutaneous, parenteral, intravenous, intranasal, topical, transdermal, intraperitoneal, intramuscular, intrapulmonary, vaginal, rectal, or intraocular administration. In some embodiments, the compounds and compositions described herein are administered orally or intravenously. In some embodiments, the compounds and compositions described herein is administered orally.

Pharmaceutically acceptable compositions include solid, semi-solid, liquid and aerosol dosage forms, such as tablet, capsule, powder, liquid, suspension, suppository, and aerosol forms. The compounds and compositions described herein can also be administered in sustained or controlled release dosage forms (e.g., controlled/sustained release pill, depot injection, osmotic pump, or transdermal (including electrotransport) patch forms) for prolonged timed, and/or pulsed administration at a predetermined rate. In some embodiments, the compositions are provided in unit dosage forms suitable for single administration of a precise dose.

The compounds and compositions described herein can be administered either alone or in combination with one or more conventional pharmaceutical carriers or excipients (e.g., mannitol, lactose, starch, magnesium stearate, sodium saccharine, talcum, cellulose, sodium crosscarmellose, glucose, gelatin, sucrose, magnesium carbonate). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate). Generally, depending on the intended mode of administration, the pharmaceutical composition will contain about 0.005% to 95%, or about 0.5% to 50%, by weight of a compound disclosed and/or described herein. Actual methods of preparing such dosage forms are known, or will be apparent, to those skilled in this art; for example, see Remington’s Pharmaceutical Sciences, Mack Publishing Company, Easton, Pennsylvania.

In some embodiments, the compositions will take the form of a pill or tablet and thus the composition may contain, along with the compounds described herein, one or more of a diluent (e.g., lactose, sucrose, dicalcium phosphate), a lubricant (e.g., magnesium stearate), and/or a binder (e.g., starch, gum acacia, polyvinylpyrrolidine, gelatin, cellulose, cellulose derivatives). Other solid dosage forms include a powder, marume, solution or suspension (e.g., in propylene carbonate, vegetable oils or triglycerides) encapsulated in a gelatin capsule.

Liquid pharmaceutically administrable compositions can, for example, be prepared by dissolving, dispersing or suspending etc. the compounds described herein and optional pharmaceutical additives in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of the compounds contained in such parenteral compositions depends, for example, on the physical nature of the compounds, the activity of the compounds, and the needs of the subject. However, percentages of active ingredient of 0.01% to 10% in solution are employable, and may be higher if the composition is a solid which will be subsequently diluted to another concentration. In some embodiments, the composition will comprise from about 0.2 to 2% of a compound described herein in solution.

Pharmaceutical compositions of the compounds and compositions described herein may also be administered to the respiratory tract as an aerosol or solution for a nebulizer, or as a microfine powder for insufflation, alone or in combination with an inert carrier such as lactose. In such a case, the particles of the pharmaceutical composition may have diameters of less than 50 microns, or in some embodiments, less than 10 microns.

In addition, pharmaceutical compositions can include compounds described herein and one or more additional medicinal agents, pharmaceutical agents, adjuvants, and the like.

### Kits

Also provided are articles of manufacture and kits containing any of the compounds and compositions provided herein. The article of manufacture may comprise a container with a label. Suitable containers include, for example, bottles, vials, and test tubes. The containers may be formed from a variety of materials such as glass or plastic. The container may hold a pharmaceutical composition provided herein. The label on the container may indicate that the pharmaceutical composition is used for preventing, treating or suppressing a condition described herein, and may also indicate directions for either in vivo or in vitro use.

In one aspect, provided herein are kits containing the compounds and compositions described herein and instructions for use. The kits may contain instructions for use in the treatment of any disease provided herein in a subject in need thereof. A kit may additionally contain any materials or equipment that may be used in the administration of the compounds and compositions, such as vials, syringes, or IV bags. A kit may also contain sterile packaging.

### ENUMERATED EMBODIMENTS

1. A compound of Formula (I),

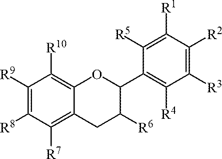

or a pharmaceutically acceptable salt, stereoisomer or tautomer thereof, wherein:
R¹, R², R³, R⁴ and R⁵ are independently H, OH, halo, C₁-C₆ alkyl, or C₁-C₆ alkoxy, wherein at least one of R¹, R⁴ and R⁵ is OH, halo, C₁-C₆ alkyl, or C₁-C₆ alkoxy;
R⁶ is OH or H; and
R⁷, R⁸, R⁹ and R¹⁰ are independently H, OH, C₁-C₆ alkyl, or C₁-C₆ alkoxy,
provided that the compound is not selected from the group consisting of:
2-(3,4-dihydroxyphenyl)chroman-3,7-diol,
2-(3,4-dihydroxyphenyl)chroman-3,5-diol,
2-(3-hydroxyphenyl)chroman-3,5,7-triol,
2-(3,4-dihydroxy-2-methylphenyl)chroman-3,5,7-triol,
2-(2-fluoro-3,4-dihydroxyphenyl)chroman-3,5,7-triol,
2-(2-fluoro-4,5-dihydroxyphenyl)chromane-3,5,7-triol,
2-(3-fluoro-4-hydroxyphenyl)chromane-3,5,7-triol,
2-(3,4-dihydroxy-5-methylphenyl)chromane-3,5,7-triol,
2-(3,4-dihydroxyphenyl)chromane-3,5,7-triol,
4-(3-hydroxychroman-2-yl)benzene-1,2-diol,
2-(3-hydroxyphenyl)chromane-3,5-diol,
2-(3-hydroxyphenyl)chromane-3,7-diol,
2-(3-hydroxyphenyl)chroman-3-ol,
2-(3-methoxyphenyl)chromane-3,7-diol,
2-(3-hydroxyphenyl)-7-methoxychroman-3-ol,
Cis (±) 7-methoxy-2-(3-methoxyphenyl)chroman-3-ol,
Cis (±) 7-methoxy-2-(4-methoxyphenyl)chroman-3-ol,
2-(3-methoxy-4-methylphenyl)chromane-3,7-diol,
2-(3-hydroxy-4-methylphenyl)chromane-3,7-diol,
2-(4-fluoro-3-methoxyphenyl)chromane-3,7-diol,
2-(4-fluoro-3-hydroxyphenyl)chromane-3,7-diol,
2-(3,4-dihydroxyphenyl)chroman-3,7-diol,
2-(3,4-dihydroxyphenyl)chroman-3,5-diol,
2-(3-ethoxy-4-hydroxyphenyl)chromane-3,5,7-triol,
4-(3-hydroxy-5,7-dimethoxychroman-2-yl)benzene-1,2-diol,
2-(4-hydroxy-3-propoxyphenyl)chromane-3,5,7-triol,
2-(4-ethoxy-3-hydroxyphenyl)chromane-3,5,7-triol,
2-(3-hydroxy-4-propoxyphenyl)chromane-3,5,7-triol, and
2-(4-hydroxy-3-methoxyphenyl)chromane-3,5,7-triol,
or a pharmaceutically acceptable salt, stereoisomer or tautomer thereof.

2. The compound of embodiment 1, or a pharmaceutically acceptable salt, stereoisomer or tautomer thereof, wherein the substitution at C2 and C3 of the pyran ring is cis(+) or cis(-) or a mixture thereof.

3. The compound of embodiment 1 or 2, or a pharmaceutically acceptable salt, stereoisomer or tautomer thereof, wherein R⁶ is OH.

4. The compound of any one of embodiments 1-3, or a pharmaceutically acceptable salt, stereoisomer or tautomer thereof, wherein R¹, R², R³, R⁴ and R⁵ are independently H, OH, F, Cl, CH₃, CF₃, or OCH₃.

5. The compound of any one of embodiments 1-4, or a pharmaceutically acceptable salt, stereoisomer or tautomer thereof, wherein R⁷, R⁸, R⁹ and R¹⁰ are independently H, OH, CH₃, or OCH₃.

6. The compound of any one of embodiments 1-5, or a pharmaceutically acceptable salt, stereoisomer or tautomer thereof, wherein R⁷ and R⁹ are independently OH or OCH₃, and R⁸ and R¹⁰ are H.

7. The compound of embodiment 1, or a pharmaceutically acceptable salt, stereoisomer or tautomer thereof, wherein the compound is of formula (I-a).

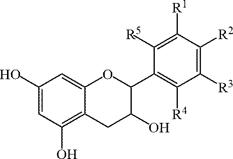

wherein the substitution at C2 and C3 of the pyran ring is cis(+) or cis(-) or a mixture thereof.

8. A compound, or a pharmaceutically acceptable salt, stereoisomer or tautomer thereof, wherein the compound is selected from the group consisting of:

| Compound | Name |
|---|---|
| 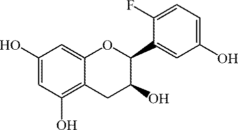 [SMILES: Oc1cc(O)c2c(c1)O[C@@H](c1cc(O)ccc1F)[C@@H](O)C2] | (2S,3S)-2-(2-fluoro-5-hydroxyphenyl)chromane-3,5,7-triol |
| 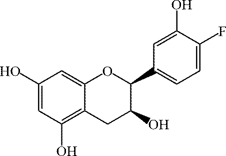 [SMILES: Oc1cc(O)c2c(c1)O[C@@H](c1ccc(F)c(O)c1)[C@@H](O)C2] | (2S,3S)-2-(4-fluoro-3-hydroxyphenyl)chromane-3,5,7-triol |
| 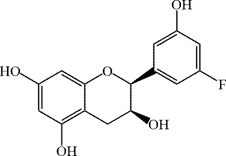 [SMILES: Oc1cc(F)cc([C@@H]2Oc3cc(O)cc(O)c3C[C@@H]2O)c1] | (2S,3S)-2-(3-fluoro-5-hydroxyphenyl)chromane-3,5,7-triol |
| 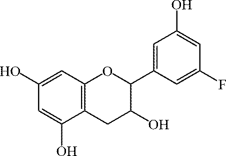 [SMILES: Oc1cc(F)cc(C2Oc3cc(O)cc(O)c3CC2O)c1] | 2-(3-fluoro-5-hydroxyphenyl)chromane-3,5,7-triol |
| 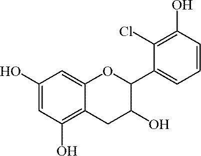 [SMILES: Oc1cc(O)c2c(c1)OC(c1cccc(O)c1Cl)C(O)C2] | 2-(2-chloro-3-hydroxyphenyl)chromane-3,5,7-triol |
| 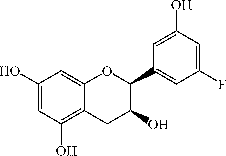 [SMILES: Oc1cc(F)cc([C@@H]2Oc3cc(O)cc(O)c3C[C@@H]2O)c1] | (2S,3S)-2-(3-fluoro-5-hydroxyphenyl)chromane-3,5,7-triol |
| 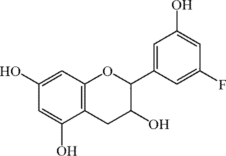 [SMILES: Oc1cc(F)cc(C2Oc3cc(O)cc(O)c3CC2O)c1] | 2-(3-fluoro-5-hydroxyphenyl)chromane-3,5,7-triol |
| 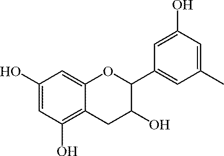 [SMILES: Cc1cc(O)cc(C2Oc3cc(O)cc(O)c3CC2O)c1] | 2-(3-hydroxy-5-methylphenyl)chromane-3,5,7-trio |
| 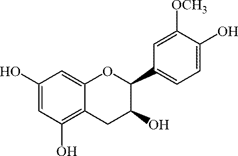 [SMILES: COc1cc([C@@H]2Oc3cc(O)cc(O)c3C[C@@H]2O)ccc1O] | (2S,3S)-2-(4-hydroxy-3-methoxyphenyl)chromane-3,5,7-triol |
| 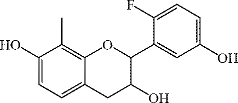 [SMILES: Cc1c(O)ccc2c1OC(c1cc(O)ccc1F)C(O)C2] | 2-(2-fluoro-5-hydroxyphenyl)-8-methylchromane-3,7-diol |
| 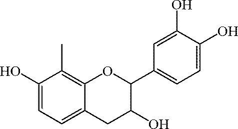 [SMILES: Cc1c(O)ccc2c1OC(c1ccc(O)c(O)c1)C(O)C2] | 2-(3,4-dihydroxyphenyl)-8-methylchromane-3,7-diol |

, or a pharmaceutically acceptable salt, stereoisomer or tautomer thereof.

9. A pharmaceutical composition comprising a compound of any one of embodiments 1-8, a pharmaceutically acceptable salt, stereoisomer or tautomer thereof, and a pharmaceutically acceptable carrier.

10. A compound, or a pharmaceutically acceptable salt, stereoisomer or tautomer thereof, of any one of embodiments 1-8 for use in AMPK activation.

Certain specific aspects and embodiments will be explained in more detail with reference to the following examples, which are provided only for purposes of illustration and should not be construed as limiting the scope in any manner.

### EXAMPLES

### Example 1: Preparation of (2S,3S)-2-(4-Hydroxy-3-Methoxyphenyl)Chromane-3,5,7-Triol

(2S,3S)-2-(4-hydroxy-3-methoxyphenyl)chromane-3,5,7-triol was prepared and characterized. MS (ESI) calculated mass: 304.00 [M]⁺, observed m/z: 304.9 [M +1]⁺; HPLC purity: 99 %, Chiral RT 5.516, ee >98 %.¹H NMR (300 MHz, DMSO-d6): 8/ppm 9.20 (s, 1H), 8.97-8.92 (m, 2H), 7.09-7.08 (d, J = 3 Hz, 1H), 6.87-6.77 (m ,2H), 5.96-5.95 (d, J = 3 Hz, 1H), 5.79-5.78 (d, J = 3 Hz, 1H), 4.85 (s, 1H), 4.76-4.75 (s, J = 3 Hz, 1H), 4.10-4.09 (bd, 1H), 3.80 (s, 3H),2.78-2.71 (m, 2H).

### Example 2: PGC-1α Assay

Culture conditions: DMEM containing 25 mM glucose with 10 % FBS supplementation; grown at 37° C. and 5 % CO₂.

Assay Conditions: C2C12 cells were cultured in DMEM supplemented in 10 % FBS up to 80 % confluence. The cells were further trypsinized and seeded into a 96 well plate at a density of 5000 cells/well (well volume – 100 µL) (Day 0). Following attachment, the cells were allowed to reach 80-90% confluence in the plate (typically 48 hours post seeding) and further differentiated using DMEM + 2 % Horse Serum (200 µL/well). The media was changed every day or at least every alternate day for 5 days to allow myoblasts to differentiate into myotubes. On day 7 (post seeding), the cells were treated with standards and test compounds for PGC-1α assay.

PGC-1α assay: For the detection of PGC-1α, the cells were incubated with 0.5 µg/ml primary antibody (Merck, WH0010891M3) in PBS-T containing 5 % BSA at 4° C. overnight. The cells were then washed three times with PBS-T for 5 minutes and incubated with 1:1000 dilution secondary antibody (Anti- rabbit IgG, HRP-linked Antibody, Cell Signaling) in PBS-T with 1 % BSA for 1 hour at RT. Cells were washed three times with PBS-T for 5 minutes The cells were incubated with 100 µl TMB substrate solution for 30 minutes and the reaction was stopped with 100 µl of 2N H₂SO₄. Then the plate was read at 450 nM using ELISA plate reader and absorbance recorded. % activity was calculated using DMSO control as 100 %. (For plate optical density calculations, a background correction was taken at 540 nm). Each compound was tested at a concentration of 0.01 nM. The results of the assay are presented in Table 2.

| Compound | Single point PGC1-Alpha Assay: Activity (%) | Single point PGC1-Alpha Assay: Average Activity (%) | Single point PGC1-Alpha Assay: Average Activity (%) Count |
|---|---|---|---|
| 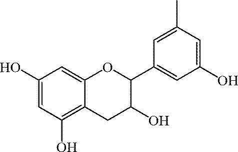 [SMILES: Cc1cc(O)cc(C2Oc3cc(O)cc(O)c3CC2O)c1] | 109 | 109 | 1 |
| 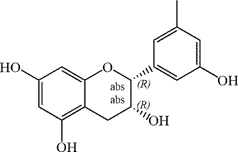 [SMILES: Cc1cc(O)cc([C@H]2Oc3cc(O)cc(O)c3C[C@H]2O)c1] | 103 | 103 | 1 |
| 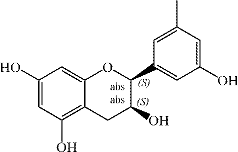 [SMILES: Cc1cc(O)cc([C@@H]2Oc3cc(O)cc(O)c3C[C@@H]2O)c1] | 114 | 114 | 1 |
| 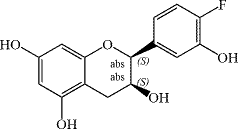 [SMILES: Oc1cc(O)c2c(c1)O[C@@H](c1ccc(F)c(O)c1)[C@@H](O)C2] | 104 | 104 | 1 |
| 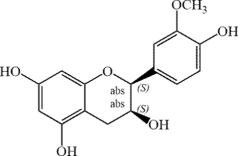 [SMILES: COc1cc([C@@H]2Oc3cc(O)cc(O)c3C[C@@H]2O)ccc1O] | 124 | 124 | 1 |
| 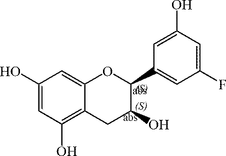 [SMILES: Oc1cc(F)cc([C@@H]2Oc3cc(O)cc(O)c3C[C@@H]2O)c1] | 117 | 117 | 1 |

1. A compound of Formula (I), 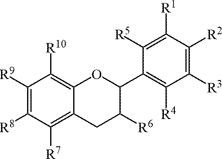
or a pharmaceutically acceptable salt, stereoisomer or tautomer thereof, wherein:
R¹, R², R³, R⁴ and R⁵ are independently H, OH, halo, C₁-C₆ alkyl, or C₁-C₆ alkoxy, wherein at least one of R¹, R⁴ and R⁵ is OH, halo, C₁-C₆ alkyl, or C₁-C₆ alkoxy;
R⁶ is OH or H; and
R⁷, R⁸, R⁹ and R¹⁰ are independently H, OH, C₁-C₆ alkyl, or C₁-C₆ alkoxy, provided that the compound is not selected from the group consisting of:
2-(3,4-dihydroxyphenyl)chroman-3,7-diol,
2-(3,4-dihydroxyphenyl)chroman-3,5-diol,
2-(3 -hydroxyphenyl)chroman- 3,5,7-triol,
2-(3,4-dihydroxy-2-methylphenyl)chroman-3,5,7-triol,
2-(2-fluoro-3,4-dihydroxyphenyl)chroman-3,5,7-triol,
2-(2-fluoro-4,5-dihydroxyphenyl)chromane-3,5,7-triol,
2-(3-fluoro-4-hydroxyphenyl)chromane-3,5,7-triol,
2-(3,4-dihydroxy-5-methylphenyl)chromane-3,5,7-triol,
2-(3,4-dihydroxyphenyl)chromane-3,5,7-triol,
4-(3-hydroxychroman-2-yl)benzene-1,2-diol,
2-(3-hydroxyphenyl)chromane-3,5-diol,
2-(3-hydroxyphenyl)chromane-3,7-diol,
2-(3-hydroxyphenyl)chroman-3-ol,
2-(3-methoxyphenyl)chromane-3,7-diol,
2-(3-hydroxyphenyl)-7-methoxychroman-3-ol,
Cis (±) 7-methoxy-2-(3-methoxyphenyl)chroman-3-ol,
Cis (±) 7-methoxy-2-(4-methoxyphenyl)chroman-3-ol,
2-(3-methoxy-4-methylphenyl)chromane-3,7-diol,
2-(3-hydroxy-4-methylphenyl)chromane-3,7-diol,
2-(4-fluoro-3-methoxyphenyl)chromane-3,7-diol,
2-(4-fluoro-3-hydroxyphenyl)chromane-3,7-diol,
2-(3,4-dihydroxyphenyl)chroman-3,7-diol,
2-(3,4-dihydroxyphenyl)chroman-3,5-diol,
2-(3-ethoxy-4-hydroxyphenyl)chromane-3,5,7-triol,
4-(3-hydroxy-5,7-dimethoxychroman-2-yl)benzene-1,2-diol,
2-(4-hydroxy- 3 -propoxyphenyl)chromane- 3,5,7 -triol,
2-(4-ethoxy-3-hydroxyphenyl)chromane-3,5,7-triol,
2-(3-hydroxy-4-propoxyphenyl)chromane-3,5,7-triol, and
2-(4-hydroxy-3-methoxyphenyl)chromane-3,5,7-triol,
or a pharmaceutically acceptable salt, stereoisomer or tautomer thereof.

2. The compound of claim 1, or a pharmaceutically acceptable salt, stereoisomer or tautomer thereof, wherein the substitution at C2 and C3 of the pyran ring is cis(+) or cis(-) or a mixture thereof.

3. The compound of claim 1, or a pharmaceutically acceptable salt, stereoisomer or tautomer thereof, wherein R⁶ is OH.

4. The compound of claim 1, or a pharmaceutically acceptable salt, stereoisomer or tautomer thereof, wherein R¹, R², R³, R⁴ and R⁵ are independently H, OH, F, Cl, CH₃, CF₃, or OCH₃.

5. The compound of claim 1, or a pharmaceutically acceptable salt, stereoisomer or tautomer thereof, wherein R⁷, R⁸, R⁹ and R¹⁰ are independently H, OH, CH₃, or OCH₃.

6. The compound claim 5, or a pharmaceutically acceptable salt, stereoisomer or tautomer thereof, wherein R⁷ and R⁹ are independently OH or OCH₃, and R⁸ and R¹⁰ are H.

7. The compound of claim 1, or a pharmaceutically acceptable salt, stereoisomer or tautomer thereof, wherein the compound is of formula (I-a). 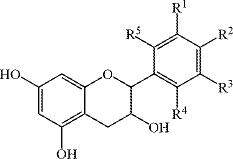
wherein the substitution at C2 and C3 of the pyran ring is cis(+) or cis(-) or a mixture thereof.

8. A compound, or a pharmaceutically acceptable salt, stereoisomer or tautomer thereof, wherein the compound is selected from the group consisting of:

| Compound | Name |
|---|---|
| 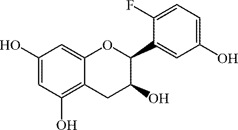 [SMILES: Oc1cc(O)c2c(c1)O[C@@H](c1cc(O)ccc1F)[C@@H](O)C2] | (2S,3S)-2-(2-fluoro-5-hydroxyphenyl)chromane-3,5,7-triol |
| 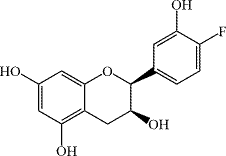 [SMILES: Oc1cc(O)c2c(c1)O[C@@H](c1ccc(F)c(O)c1)[C@@H](O)C2] | (2S,3S)-2-(4-fluoro-3-hydroxyphenyl)chromane-3,5,7-triol |
| 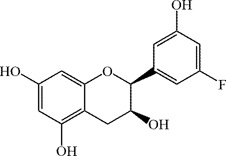 [SMILES: Oc1cc(F)cc([C@@H]2Oc3cc(O)cc(O)c3C[C@@H]2O)c1] | (2S,3S)-2-(3-fluoro-5-hydroxyphenyl)chromane-3,5,7-triol |
| 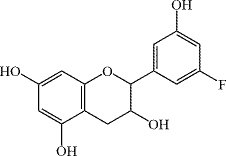 [SMILES: Oc1cc(F)cc(C2Oc3cc(O)cc(O)c3CC2O)c1] | 2-(3-fluoro-5-hydroxyphenyl)chromane-3,5,7-triol |
| 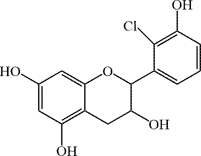 [SMILES: Oc1cc(O)c2c(c1)OC(c1cccc(O)c1Cl)C(O)C2] | 2-(2-chloro-3-hydroxyphenyl)chromane-3,5,7-triol |
| 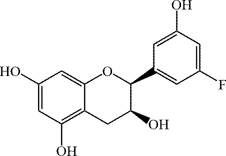 [SMILES: Oc1cc(F)cc([C@@H]2Oc3cc(O)cc(O)c3C[C@@H]2O)c1] | (2S,3S)-2-(3-fluoro-5-hydroxyphenyl)chromane-3,5,7-triol |
| 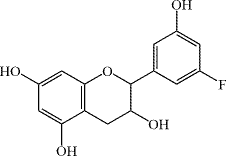 [SMILES: Oc1cc(F)cc(C2Oc3cc(O)cc(O)c3CC2O)c1] | 2-(3-fluoro-5-hydroxyphenyl)chromane-3,5,7-triol |
| 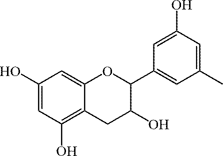 [SMILES: Cc1cc(O)cc(C2Oc3cc(O)cc(O)c3CC2O)c1] | 2-(3-hydroxy-5-methylphenyl)chromane-3,5,7-trio |
| 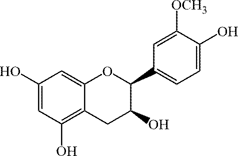 [SMILES: COc1cc([C@@H]2Oc3cc(O)cc(O)c3C[C@@H]2O)ccc1O] | (2S,3S)-2-(4-hydroxy-3-methoxyphenyl)chromane-3,5,7-triol |
| 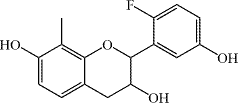 [SMILES: Cc1c(O)ccc2c1OC(c1cc(O)ccc1F)C(O)C2] | 2-(2-fluoro-5-hydroxyphenyl)-8-methylchromane-3,7-diol |
| 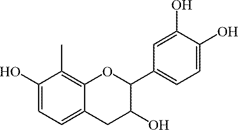 [SMILES: Cc1c(O)ccc2c1OC(c1ccc(O)c(O)c1)C(O)C2] | 2-(3,4-dihydroxyphenyl)-8-methylchromane-3,7-diol |

, or a pharmaceutically acceptable salt, stereoisomer or tautomer thereof.

9. A pharmaceutical composition comprising a compound of claim 1, a pharmaceutically acceptable salt, stereoisomer or tautomer thereof, and a pharmaceutically acceptable carrier.

10. A compound, or a pharmaceutically acceptable salt, stereoisomer or tautomer thereof, of claim 1, for use in AMPK activation.
